(19) United States
(12) Patent Application Publication
(10) Pub. No.: US 2026/0126432 A1
(43) Pub. Date: May 7, 2026

(54) POINT OF CARE IMMUNIZATION TESTING SYSTEM-DETECTION METHODS

(71) Applicant: ImmunoProfile, LLC, East Brunswick, NJ (US)

(72) Inventor: Sudip GHATAK, East Brunswick, NJ (US)

(21) Appl. No.: 19/426,505

(22) Filed: Dec. 19, 2025

Related U.S. Application Data
(63) Continuation of application No. 18/307,192, filed on Apr. 26, 2023, now abandoned.
Application No. 18/307,192 is a continuation of application No. 15/978,751, filed on May 14, 2018, now abandoned.
Application No. 15/978,751 is a continuation of application No. 15/599,775, filed on May 19, 2017, now Pat. No. 10,788,482.
Application No. 15/599,775 is a division of application No. 14/360,763, now abandoned.
(60) Provisional application No. 61/563,887, filed on Nov. 28, 2011.

(51) Int. Cl. G01N 33/53; G01N 33/68

(52) U.S. Cl. CPC G01N 33/53; G01N 33/6854

(57) ABSTRACT

A point of care immunization system based upon a range of detection methods to rapidly test a patient in order to ascertain an immunization profile so that vaccinations can be administered to address identified gaps. A point of care system comprised of sample and test strips/cartridges, with said test strips/cartridges configured to meet healthcare requirements of national governing bodies. A point of care system is provided that can function as a stand-alone diagnostic system, a stand-alone kit or with a device.

### CROSS-REFERENCE TO RELATED APPLICATIONS

This application is a continuation U.S. patent application Ser. No. 18/307,192 filed Apr. 26, 2023 which is a continuation of U.S. patent application Ser. No. 15/978,751 filed May 14, 2018, which is a continuation of U.S. patent application Ser. No. 15/599,775, filed May 19, 2017, which is a divisional of U.S. patent application Ser. No. 14/360,763, filed May 27, 2014, which is the U.S. National Phase of International Application Serial No. PCT/US2012/066226, filed Nov. 21, 2012, which claims the benefit of U.S. Provisional Application Ser. No. 61/563,887, filed Nov. 28, 2011; U.S. patent application Ser. No. 15/978,751 is also a continuation of the U.S. patent application Ser. No. 14/749,044 filed Jun. 24, 2015, which claims the benefit of U.S. Provisional Application Ser. No. 62/016,975 filed Jun. 25, 2014; U.S. patent application Ser. No. 15/978,751 is also a continuation of the U.S. patent application Ser. No. 14/619,435 filed Feb. 11, 2015, which claims the benefit of U.S. Provisional Application Ser. No. 61/938,482 filed Feb. 11, 2014, which entire contents of each of which are incorporated by reference herein.

This application is also a continuation of U.S. patent application Ser. No. 14/749,044, filed Jun. 24, 2015, which claims the benefit of U.S. Provisional Patent Application Ser. No. 62/016,975, filed Jun. 25, 2014, the entire contents of each of which are incorporated by reference herein. This application is also a continuation of U.S. patent application Ser. No. 14/619,435, filed Feb. 11, 2015, which claims the benefit of U.S. Provisional Patent Application Ser. No. 61/938,482, filed Feb. 11, 2014, the entire contents of each of which are incorporated by reference herein.

### TECHNICAL FIELD

The present disclosure relates to, among other things, a system to diagnose a patient's immunization protection levels.

### BACKGROUND

Point of care diagnostic instruments have been employed for years in medical offices and clinical settings to target various applications. Typically, point of care instruments are targeted at rapid testing to detect a patient's exposure to an infectious agent or to provide general information on vital statistics such red blood cell count, white blood cell count and amount of lead present. Infectious disease tests include situations such as determining the presence of streptococcal (strep) bacteria in diagnosing strep throat. Many of these tests fall in the category of CLIA (Clinical Laboratory Improvement Amendments) waived tests, which are defined as simple laboratory examinations and procedures that are cleared by the Food and Drug Administration (FDA) for home use; employ methodologies that are so simple and accurate as to render the likelihood of erroneous results negligible; or pose no reasonable risk of harm to the patient if the test is performed incorrectly.

The main problem is that no point of care device exists to quickly and inexpensively detect a patient's antibody levels to determine immunization protections. Currently, to make such a diagnosis the patient must have blood drawn at a clinical setting and the blood sample tested in a clinical laboratory. The tests are ordered on an individual basis, such as measles, and can take more than a week for response time at high “per test” costs. If a patient needs to be tested for multiple immunization levels, separate and individual tests must be ordered. There is no availability to quickly diagnose a patient's immunity level across the recommended healthcare guidelines in an easy, rapid and cost effective manner. Furthermore, poor record tracking of individual patient's immunizations can result in patients themselves generally not knowing if they are protected.

The range of vaccine efficacies is wide—some as low as 50%—, especially in patients with compromised immune systems, and there is no way to know how an individual will respond to the vaccine. Not only is the duration of protection unpredictable, but other factors can affect a vaccine's efficacy, including missed booster shots, a change in medical condition (HIV, hepatitis, obesity), age of vaccinations, age of vaccines, compromised vaccines, or the like. Certain vaccines cannot be given to children or pregnant women, or while a patient is immuno-compromised.

If a patient is administered an old or even expired vaccine, the patient may not receive full immunity. Recalls occasionally pull out problem vaccine batches, but they are not a guarantee that all vaccines are viable and effective.

Another growing problem is that patients are opting out of vaccines, so communities no longer have near 100% immunization rates. This leads to disease outbreaks in the US and other developed countries when a disease carrier enters a vulnerable population. Disease outbreaks for measles and whooping cough have occurred in the US in 2011, along with outbreaks in Europe. Given the ease of travel from vast geographies, a disease carrier can travel almost anywhere in the world in less than twenty-four hours. The only available testing for immunity is costly for the patient, and does not yield immediate results.

### SUMMARY

Accordingly, a simple but effective solution is needed to enable patients and the healthcare provider the means to quickly test for immunization protection and address any identified gaps through the timely administration of the necessary vaccines. The system needs to be easy to use, timely and targeted. It should comprise of a simple to use diagnostic instrument with ports for the sample and the targeted test cartridge. Target test cartridges are for example designed following the recommended guidelines for each country, and are for example adapted for a range of parameters such as age, gender and life changing event. The system should provide timely test results to identify the gaps in the immunization profile and quickly fill the gaps with the timely administration of the necessary vaccine(s). The system should also have a means to update and track patient's immunization records linking it to clinical information systems and providing an electronic/paper copy to the patient.

Conventional point of care devices are better suited for disease detection and measure vital parameters from blood and other bodily fluids. Currently no point of care device is targeted for detecting immunization levels leaving most patients unclear of how well they are protected against disease until disease sets upon them.

This disclosure generally provides a way to diagnose anti-body levels for immunity levels, using a point of care diagnostic device, targeted test cartridges, corresponding vaccine supply packs, sensors for temperature, sensors of expiry, tags to establish pedigree and/or smart chips to instruct test protocols.

In accordance with one aspect, the present disclosure is directed to the diagnosis of antibody levels in a patient's sample to determine a patient immunization protection levels or allergy indicative IgE levels. (Any reference herein to “immunization testing” includes allergic IgE testing.) The disclosure is comprised of cartridges, including targeted test cartridges, with controls so that two distinct cartridges cannot be mistaken for each other. The targeted test cartridge can be configured with the necessary immunization tests per the healthcare guidelines of the specific country for the appropriate group by age, gender, life changing event, and the like. The targeted test cartridge can include a smart chip (e.g., transponder) or bar code to instruct the point of care diagnostic device to initiate the appropriate test protocols. The corresponding vaccine supply packs can be configured to the targeted test cartridge so that in the event a gap is identified in the immunization profile, the appropriate vaccine can be administered in a timely manner. The vaccine supply pack can organize the vaccine vials in an easy to store and access manner. The supply pack can include sensors for temperature detection in the event the vaccines were compromised due to refrigeration malfunction or power outages. The supply pack can include sensors providing warnings in the event a vaccine has passed its expiration preventing its use, and communication hardware for linking the sensors to supply/inventory management systems. The supply pack can also incorporate tags (IDs) such RFID (radio-frequency identification) or bar codes to establish vaccine pedigree to prevent them from being compromised or counterfeited. In certain embodiments, the disclosure provides a new and improved point of care testing device to detect antibody levels per immunization protection.

In certain embodiments, the disclosure provides a biological sample cartridge for ease of use and prevents confusion with the targeted test cartridge. The biological sample cartridge may contain anti-coagulants in the case of blood samples to facilitate the sample prep process, enable easier transfer of the blood sample and improve assay performance.

In certain embodiments, the disclosure provides a targeted test cartridge customized to the meet the healthcare requirements for specific countries across age, gender and life changing events among others.

In certain embodiments, the disclosure provides a vaccine supply pack providing ease of use, ease of access, organized storage, space saving, sensors to detect temperature, coding to indicate expiration, sensor to detect quantities/supply, tags to establish pedigree, and the like.

In certain embodiments, the disclosure provides a means to supply the healthcare provider's patient with the necessary vaccine to fill any gaps in their individual immunization profile.

In certain embodiments, the disclosure provides a method for updating a patient's immunization record for the clinical information systems and patient's personal records.

Provided is an immunization testing device for testing a biological sample from a subject comprising: an analytical module adapted to make two or more dilutions a fluid that is or is derived from the biological sample, contact said dilutions with separate replicates of vaccine- or sensitization-indicative antigens so as to generate a signals indicative of the amount of antigen-reactive immune molecules in the biological fluid dilutions.

Provided is an immunization testing device for testing a biological sample from a subject comprising, an analytical module comprising fluidic pathways for conducting dilutions, a controller, an data output device, and one or more input ports having a conjugate input comprising a test cartridge, wherein one or more of the following obtains:

wired connectivity; wireless connectivity; interfaces with hospital/clinical information systems; interfaces with laboratory information systems; provides wired and wireless printer ports; provides links to electronic patient records; provides self-maintenance (e.g., via diagnostic hardware and software for the instrument and/or the cartridges); provides links to smart phones, PDAs printers, and the like; provides biological fluid sample cartridges with:
unique shape and/or distinguishable color;
ability to supply needed sample for assay; provides targeted test cartridges with
unique shape and/or distinguishable color and/or ability to supply needed antigens for assay and/or chip to instruct instrument to initiate protocols and/or configured to meet testing requirements as set by healthcare governing bodies and/or individual chambers for each vaccine target with requisite antigen; provides vaccine supply pack with
compact storage for vaccines and/or easy access and/or small profile saving space in refrigerator and/or smart sensors to indicate temperature and/or smart sensors to indicate expiration date and/or smart sensors to indicate vaccine supply and/or linkages to supply management and ordering systems and/or radio frequency tags to establish pedigree and/or bar code tags to establish pedigree; provides smart app with
smart phones and computer devices; ability to track and update personal immunization records; ability to share personal immunization records provides business model for selling vaccines with
companion diagnostic system for vaccines and/or means for identifying gaps in a person's immunization profile and/or administering only necessary vaccines and/or charging a service or handling fee for vaccine administration and/or charging a fee for the vaccination itself.

Also provided is an immunization testing device for testing a biological sample from a subject comprising, an analytical module comprising a controller, an data output device, and one or more input ports having a conjugate input comprising a test cartridge port, wherein the test cartridge port has an ID receiver for receiving an ID from a immunization test cartridge, wherein the controller is adapted to operate the analytical module to make two or more dilutions of the biological sample, contact said dilutions with separate replicates of vaccine- or sensitization-indicative antigens so as to generate a signals indicative of the amount of antigen-reactive immune molecules in the biological dilutions, interpret the received ID to identify one of a pre-set plurality of available immunization test cartridges, and to utilize the generated signals and the immunization test cartridge to output a report on the immune status of the subject with respect to an array of separate vaccine- or sensitization-indicative antigens (e.g., 4 or more). The ports can be shaped to accept their conjugate input and not accept the conjugate inputs of other ports present. The biological sample cartridge may contain anti-coagulants in the case of blood samples to facilitate the sample prep process, enable easier transfer of the blood sample and better assay performance. The first step of the immunoprofile assay is a sample prep step to ensure the blood sample is properly managed in order to serve the purposes of the assay. Part of this effort can be, as needed, to treat the blood sample with anti-coagulant to prevent the blood from clotting which in some instances can have negative effects on the assay process.

In certain embodiments, the controller is adapted to operate with (A) a collection of immunization test cartridges comprising reagents for testing immune status against an array of vaccine- or sensitization-indicative antigens, the collection including two or more cartridges for testing separate arrays of vaccine- or sensitization-indicative antigens, the separate arrays adapted for use with separate patient populations, the cartridges having IDs that are distinctive of the separate arrays, wherein the controller reads the ID of a given utilized test cartridge and presents the output report correlating the vaccination status results with the respective vaccine- or sensitization-indicative antigens based on the read ID. In one embodiment, the controller is adapted to operate with packs of immunization compositions, separate packs matching the separate arrays of the immunization test cartridges (vaccines matching the vaccine-indicative antigens), the distinct immunization compositions identifiably spatially segregated on the packs, the packs having IDs that are distinctive of the separate arrays of immunization compositions, wherein after presenting an output report the controller compares the ID of a presented pack with the ID of the utilized test cartridge to confirm that the cognate pack has been presented. In one embodiment, the controller is adapted to operate with packs of immunization compositions, separate packs matching the separate arrays of the immunization test cartridges; wherein after presenting an output report the controller compares an ID of a presented immunization composition with the output report data to confirm that the immunization composition matches the tested vaccine-indicative antigens. In another embodiment, the controller further confirms that the presented immunization composition matches a vaccine-indicative antigen found to have a deficient immune response.

In certain embodiments, the immunization testing device takes the form of a handheld sampling device, as described below. In certain embodiments, the vaccine indicative antigens are arrayed on a test strip adapted to contact the dilutions by flow of the dilution material through columns of the test strip so as to serially contact the antigens. In certain embodiments, the test strips are incorporated into the immunization test cartridges.

Also provided is a collection of immunization test cartridges comprising reagents for testing immune status against an array of vaccine- or sensitization-indicative antigens, the collection including two or more cartridges for testing separate arrays of vaccine- or sensitization-indicative antigens, the separate arrays adapted for use with separate patient populations, the cartridges having IDs that are distinctive of the separate arrays. Further provided is a kit comprising (A) a collection of immunization test cartridges, and (B) packs of immunization compositions, separate packs matching the separate arrays of the immunization test cartridges, the distinct immunization compositions identifiably spatially segregated on the packs, the packs having IDs that are distinctive of the separate arrays of immunization compositions, the collections and packs adapted to be operative with an analytical module that utilizes the immunization test cartridges to provide subject immune statuses for the antigens of given immunization test cartridges, and which compares the ID of a utilized immunization test cartridge and that of a presented pack to confirm that the cognate pack has been presented.

In certain embodiments, the immunization composition packs comprise a temperature sensor, electronic memory for tracking temperature from the sensor over time, and wherein the packs are adapted to communicate the temperature tracking to the analytical module. In certain embodiments, the immunization test cartridges are in the form of handheld sampling devices.

Further provided is a method of operating the testing device, comprising operating the testing device with a biological sample from a patient utilizing an immunization test cartridge, and thereby outputting a report on the immune status of the subject with respect to an array of separate vaccine- or sensitization-indicative antigens. Also provided is a method of operating the testing device operative with an immunization pack ID reader, comprising operating the testing device with a biological sample from a patient utilizing a said immunization test cartridge, and thereby outputting a report on the immune status of the subject with respect to an array of separate vaccine- or sensitization-indicative antigens; presenting a said immunization pack to the immunization pack ID reader to generate output from the testing device confirming or negating that the immunization pack is the cognate of the test cartridge. The method can further include the controller obtaining from the immunization pack information on immunization stock, calculating the further utilization implied by the immune status report, and, if needed based on this data, generating (i) a report identifying vaccine restocking needs or (ii) a purchase order to a vaccine supplier. The report or purchase order can also be sent by the controller to a user or to the vaccine supplier, such as by email or fax.

Also provided is a method of operating the testing device with an immunization composition ID reader, comprising operating the testing device with a biological sample from a patient utilizing a said immunization test cartridge, and thereby outputting a report on the immune status of the subject with respect to an array of separate vaccine- or sensitization-indicative antigens; presenting a said immunization composition to the immunization composition ID reader to generate output from the testing device confirming or negating that the immunization composition is the immunization composition called for by the immune status report. In certain embodiments of the method, the analytical module is comprised in a handheld sampling device.

The above methods can be conducted at the point-of-care, such as a physician's office, clinic, patient bedside, and the like.

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only illustrative embodiments of this disclosure and are therefore not to be considered limiting of its scope, for the disclosure may admit to other equally effective embodiments.

These and other features and advantages of embodiments the present disclosure will be fully apparent from the following description, when taken in connection with the included drawings.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only illustrative embodiments of this disclosure and are therefore not to be considered limiting of its scope, for the disclosure may admit to other equally effective embodiments.

FIG. 1 is a detailed view of sample dilution profile.

FIG. 2 is a detailed view of the diagnostic assay plate.

FIG. 3 is an overview of the point of care diagnostic device.

FIG. 4 is a detailed view of a single target test cartridge for infants based upon US Centers for Disease Control guidelines.

FIG. 5 is a detailed view of an infant vaccine supply pack based upon the corresponding infant target test cartridge.

FIG. 6 shows a controller that can be incorporated into the analytical module.

FIG. 7 is a detailed exemplification of a single target test strip for infants based upon US Centers for Disease Control guidelines.

FIGS. 8A, 8B and 8C are overviews of point of care diagnostic devices, some for example using target test strips.

FIG. 9 is an overview of the point of care diagnostic device in the form of a handheld fluid analyzer.

FIG. 10 is an overview of the point of care diagnostic device in the form of a handheld fluid analyzer, with a separable component (here illustrated as a controller).

FIG. 11 is an overview of the point of care diagnostic device in the form of a handheld fluid analyzer, with a separable component.

FIG. 12 is a cross-section of the point of care diagnostic device in the form of a handheld fluid analyzer, with a separable component.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation. For examples, elements and features can be shared between various embodiments that may operate at atmospheric pressure, or higher pressures, depending on among other things the feedstock natural gas pressure available at different locations of the device.

### DETAILED DESCRIPTION

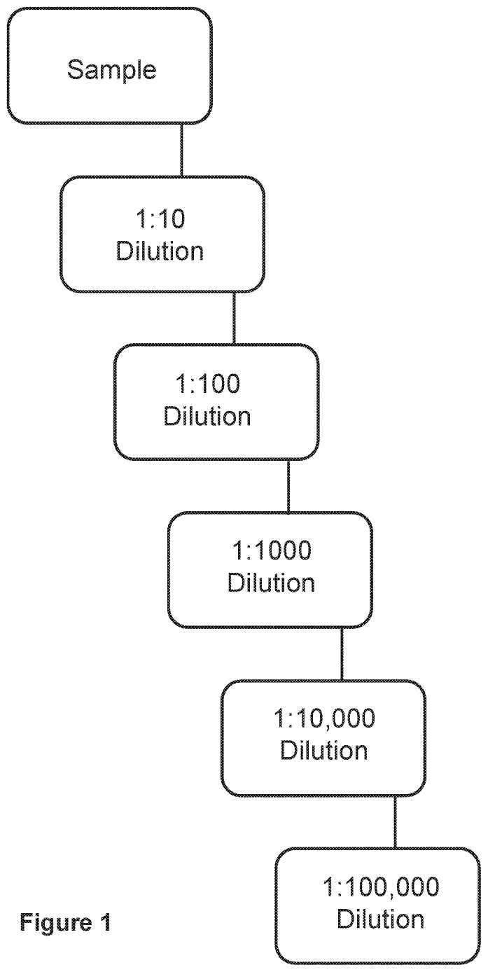

Referring now to the drawings in greater detail, FIG. 1 shows a detailed view of an exemplary sample dilution profile. The sample from the patient will be diluted per the protocols of the test procedure, typically in series fashion. Using fluidic devices (such as microfluidics or robotic pipetting) the sample can be mixed with dilution buffer solution to create the first dilution (e.g. 1:10) which is one part sample and nine parts buffer. The same or alternative dilutions can be conducted on dilutions of a given dilution step. In the Figure we illustrate a simple dilution system, but the point of care device can make virtually any dilution pool by taking the appropriate amount of sample combined with the specified amount of buffer. The figure is only an illustration of one such dilution profile.

The biological sample can be a fluid (e.g., blood, sera, lymph fluid, urine, tears, saliva or the like), a tissue (such as marrow, hair follicles, or the like). In the case of non-fluids, antibodies are extracted. The sample can be from a human, or from any animal with an immune system, such as a dog, cat, horse, donkey, elephant, manatee,

In the diagnostic method conducted in the immunization testing device, dilutions are contacted with the respective vaccine- or sensitization-indicative antigens (or control antigens) and the ancillary agents used to develop a signal indicative of the amount of antibody responsive to the antigen that is present. These ancillary agents can be antibodies, color-developing reagents (inclusive of fluorescence-developing reagents), and the like. Fluorescence can include FRET, wherein fluorescence transfer between two assay moieties enhances signal-to-noise. The diagnostic method (assay) will typically at some point fix the signal on a surface to provide a way of concentrating signal and washing away reagent that might provide false signal. The vaccine- or sensitization-indicative antigen can be provided fixed to the surface, or can be fixed during the course of the diagnostic method. The assay can be a competitive assay, in which a diminution of signal is indicative of unlabeled antibody from the biological sample competing out labeled antibody. Or, the assay can provide a positive correlation between experimental antibody amount and signal, such as via a sandwich assay. In this case, for example, label can be on antigen, on reagent antibody, or the like. Label can be directly measurable, or be the means for developing a measurable signal, such as by being reactive with a binding moiety (e.g., antibody, lectin) having a further label, or by having an attached enzyme.

A given dilution can be assayed against different antigens in separate compartments, or can be assayed such that the signals for the different antigen localize in different places. For example, where the antigen is labeled, it might be captured by different non-competitive antibody localized separately from the capture antibodies for other antigens. Or the antigen can be separately localized on a solid. For example, separate, identifiable beads or other solids can be incubated with the dilution to provide separate signals, or signal can be spatially resolved on a surface. Immuno-chromatographic methods (lateral flow) can be used to move reagents over the detection surface and provide washing. In lateral flow, reagents can be provided dry on a portion of a surface, and be solubilized and caused to flow over the signal-generating portion. In certain embodiments, diluted biological can be delivered to an absorbent region, providing a reservoir of fluid for lateral flow.

In certain embodiments, the dilution and/or the presentation and/or removal of reagents for developing signal are delivered robotically, for example using pipetting methods.

The methods and devices of the disclosure are adapted to facilitate assaying for a substantial set (e.g., four or more) of vaccination statuses in one relatively quick operation beginning with drawing a biological sample. This set can be termed a “diagnosis set.” Test cartridges are adapted to provide the all of the vaccine- or sensitization-indicative antigens of a given set. Sets are designed to be particularly useful for particular populations; for example, one set is used for preschoolers, another for male teens, another for female teens, another for travelers to a given region, and the like.

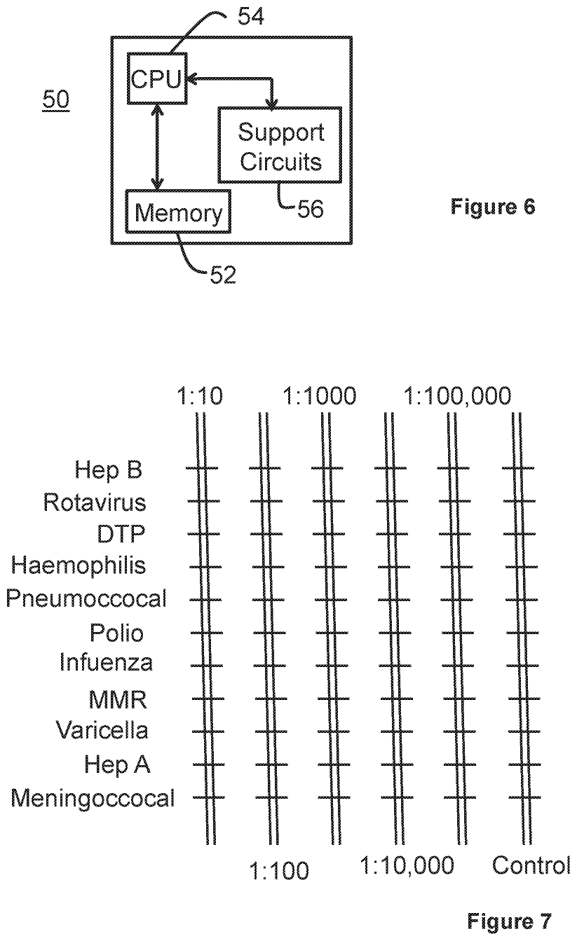

The analytical module can contain a controller 50 (FIG. 6), which comprises a central processing unit (CPU) 54, a memory 52, and support circuits 56 for the CPU 54 and is coupled to and controls the various elements of the immunization testing device or, alternatively, operates to do so in conjunction with computers (or controllers) connected to the immunization testing device. For example, another electronic device can supply software, or operations may be calculated off-sight with controller 50 coordinating off-sight operations with the local environment. The controller 50 may be one of any form of general-purpose computer processor that can be used for controlling various devices and sub-processors. The memory, or computer-readable medium, 52 of the CPU 54 may be one or more of readily available memory such as random access memory (RAM), read only memory (ROM), flash memory, floppy disk, hard disk, or any other form of digital storage, local or remote. The support circuits 56 are coupled to the CPU 54 for supporting the processor in a conventional manner. These circuits can include cache, power supplies, clock circuits, input/output circuitry and subsystems, and the like. Methods of operating the immunization testing device may be stored in the memory 52 as software routine that may be executed or invoked to control the operation of the immunization testing device 100. The software routine may also be stored and/or executed by a second CPU (not shown) that is remotely located from the hardware being controlled by the CPU 54. While the above discussion may speak of the “controller” taking certain actions, it will be recognized that it may take such action in conjunction with connected devices.

In certain embodiments, the controller is a smart phone, tablet, PC or the like that connects to the analytical module by wire connections or wirelessly.

In certain embodiments certain of the logic circuits or algorithms may be distantly external, such as in Canada. In such embodiments the “controller” is made up of the electronic elements at or near the point-of-care that coordinate data going to such distant logic circuits and operative instructions derived from such distant logic circuits.

In collecting the sample, either via the sample cartridge, test cartridge or a conventional collection tube, the collection will generally be labeled with patient identifying information. It may be that the labeling will be via a microchip or matrix code (2D bar code) that is rich in information. Thus, a data input device for the immunization testing device can be a scanner for this data rich information.

The immunization testing device may be adapted to operate in conjunction with one or a variety of devices utilized with electronic medical records, such as i-Pads, Android devices, i-Phones, laptops, and the like. In this manner, associating the testing with patient information can be facilitated, and data that must be keyboarded in (or voice to text converted) or can be inputted with a device that is better optimized for these functions. Even where significant data input and output is done via an linked device, it can be useful to have an output screen of the device, since after initiation it may prove convenient for the associated medical records device to be used with another patient in another room. More immediate input tools can also be useful, such as just a few buttons whose functions change with the circumstances as announced by for example an adjacent portion of an output screen.

Thus, the output device of the immunization testing device can be a wire output (e.g., USB), or a wireless transmitter (e.g., Bluetooth).

The test cartridge(s) are preferably one test cartridge, but in certain embodiments two or more test cartridges are used. The test cartridge(s) provide the consumable analytical reagents, and may include dilution fluid (e.g., should such not be presented by a biological sample cartridge). In certain embodiments, the test cartridge(s) provide all sample contacting materials, such as disposable pipet tips, or fluidic pathways, or reaction wells, or the like, or all such elements used downstream of biological sample dilution. Inputs (such as a test cartridge) into the analytical module can have a unique shape (such as guide features) such that it can only be connected to its cognate port. Such inputs and the port can be marked with a shared, otherwise unique color or symbol to facilitate quick connection to the correct port.

In certain embodiments, all the reagents, including the vaccine- or sensitization-indicative antigens of a diagnosis set, are provided on one test cartridge. Other test cartridges may be used to provide reagents that do not vary across analyses of diagnosis sets.

In certain embodiments, inputs or other components have IDs, which are symbols, codes (such as linear or multi-dimensional bar codes), devices responsive to electromagnetic queries to emit an electromagnetic identifier (e.g., RFID devices), circuit boards (which may connect via electrical contacts or wirelessly) or the like that can be read by reading devices on the analytical module. An ID receiver can be, among other things, electrical contacts. In this embodiment, the testing device's controller reads the ID(s) and utilizes the information as needed for conducting the analytical processing and reporting. It may be that the same processing steps are conducted for all test cartridges and all vaccine- or sensitization-indicative antigens, in which case the ID information may inform the report generated, or may inform the interpretation of a positive or negative result for a given vaccine- or sensitization-indicative antigen. (Notwithstanding the use of the singular form, a vaccine- or sensitization-indicative antigen may be a pool of antigens.)

IDs can include or imply a range of definitional information, or information that otherwise aids the controller or the user. For example, the ID for the test cartridge can include information on the target subjects, such as age, gender, geography and life changing events, or the like, or information on the cartridge manufacture date or expiration, or the like. The ID may include or imply the antigens used, there locations, and the like.

The controller can operate upon reading the IDs to relay the identifying information to the operator. For example, an output screen may announce “A Test Cartridge Infant Immunization Set Has Been Installed. Is This Correct?” A validating response may be required, as inputted by touchscreen, fixed buttons, keyboard, or the like.

Fluidics technology, which at smaller scales can be designated microfluidics technology, has existed for many years with numerous applications including clinical diagnostic devices. Examples include the methods and apparatuses of Kellogg et al., U.S. Pat. No. 7,476,361 (Microfluidics devices and methods of diluting samples and reagents), which include methods and apparatus for performing small-scaled analytic and synthetic procedures. The devices and methods utilize centripetal force resulting from rotation of a platform to motivate fluid movement through channels. Serial dilutions are provided. In another example, Schulte et al., US Patent Application No. 2004/0229378 A1 (Well plate microfluidics), discloses in devices and methods for performing a fluidic processes. The Schulte device includes a well plate comprising a plate and an array of wells formed on or in the plate and a fluidic structure connecting at least two of the wells. The device relies on gravitational and capillary forces that exist in channels within the fluidic structure when receiving fluid streams. Methods and devices for moving fluids with electrodes (electrohydrodynamically) and controlling the flow are described, for example, in U.S. Pat. Nos. 5,992,820, 6,106,685 and 6,109,717; and microfluidic methods using a pumping fluid to indirectly move fluids with electrodes are described in U.S. Pat. No. 5,961,800, the entire contents of each of these disclosures is incorporated herein by reference.

Where fluidic controls, such as electrically operated valves or electrodes, reside in a test cartridge, the cartridge can be adapted to contact electrical leads to the analytical module upon attachment of the cartridge to the module. In certain embodiments, the electrical contacts are located above the fluid moving conduits to minimize fluid contact with the electrical components.

The term dilution refers to the reduction in the amount of a particular subject material per unit volume of a fluid containing that material, through the addition of a second fluid or diluents, which dilution can be conducted serially. The diluent may take on a variety of forms, including aqueous and non-aqueous fluids and may include additional material components such as soluble chemical components or suspensions or emulsions of at least partially insoluble components. The subject material composition including chemical compounds either soluble or as suspensions or emulsions, biological material, either soluble or as a suspensions or emulsions and the like. Serial dilution means successive dilutions where the subject material is diluted with diluent to form a first diluted material, which first diluted material is then diluted with a diluent again, to produce a second diluted material, and so on. For example, one produces a first diluted material that is diluted 1:10 over the subject material. By then diluting at least a portion of this material 1:10, one produces a second dilution material that is a 1:100 dilution of the subject material. In general, the methods, devices and systems of the present disclosure are useful in subject material greater than 10 fold (1:10), typically greater than 100 fold (1:100), preferably greater than 1000 fold (1:1000) and in many cases, greater than 10,000 fold (1:10,000), within a single integrated microfluidic device, which typically has an integral volume, such as channel volume, of less than 10 ul and preferably less than 1 ul. Serial dilutions can also be made in different scales such as an 8 scale whereby the dilutions would be 1:8, 1:64, 1:512 etc. For example, a microfluidic device can inject 1 volume of the subject material and 7 volumes of diluent to a mixing chamber. Mixing can be by magnetic stirrer, ultrasonic, vortexing of the immunization test cartridge, or the like. From there, 7 volumes are available for the assay, and 1 volume can be injected into a next dilution mixing chamber for the next serial dilution.

The ability to perform serial dilutions using different scales can also enable the production of a customized scale such as 1:10, 1:64, 1:100, 1:512, 1:1000 etc, This is accomplished by selecting the appropriate amount of diluted sample and diluting it with the required amount of diluent. Methods and devices for serial dilutions and controlling the flow are described, for example, in U.S. Pat. No. 5,869,004, which is incorporated herein by reference in its entirety.

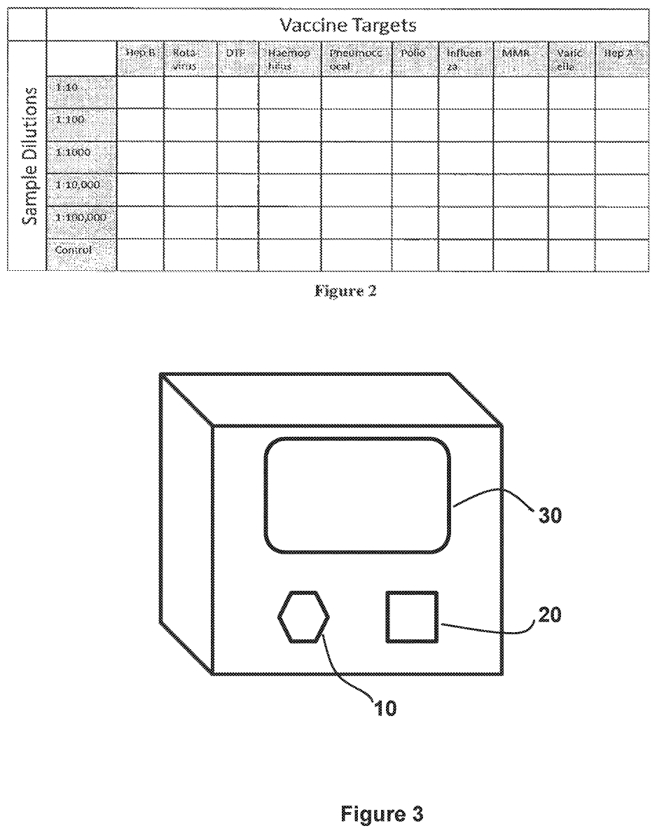

FIG. 2 shows a detailed view of an illustrative diagnostic assay plate, which in certain embodiments can be a test cartridge, or incorporated into a test cartridge. The assay plate can be populated with the prescribed dilution pool samples tested against the target antigens. Each well in the assay plate can be populated with equal amounts of antigen from the targeted test cartridge. For example, in the first column, Hep B antigen is present in each well in equal amounts. In certain embodiments, the Hep B antigen is taken from the target test cartridge and distributed in equal amounts into the designated portion of the assay plate. The sample dilutions are also introduced, generally in equal and specified volumes into each well. A color change for example will occur if the antibody is present in sufficient amount in the diluted sample upon binding with the antigen. Next the instruments analyze the sample dilutions that exhibit a signal change and the ones that do not. This data is then used to deduce a dose response curve. The patient's dose response curve is matched against a standard, such as may be designated by a healthcare agency such as the CDC (Centers for Disease Control). If the dose response curve meets the establish standard then no action is needed. If it does not match then the physician may decide to administer a vaccine or vaccine booster per the needs of the patient. This process repeats for each of the antigens in the test cartridge(s). By testing against the recommended panel of target vaccinations a patient will have a complete picture of their immunization profile and how well they are protected against disease. Gaps in the immunization profile can be easily addressed through the administration of the necessary vaccines from the vaccine supply packs.

Biological samples used with the immunization testing device are those that can include antibodies indicative of an immune response in measurable amounts. Often, the biological samples are blood or blood serum, but other biological fluids such as urine or saliva may be used. Biological fluids can be treated to remove larger or higher density elements such as RBCs, lymphocytes or platelets. Such treatment can be by filtration or centrifugation. For blood, an anticoagulant may be used during collection. In certain embodiments, such treatment is done on the immunization testing device with components provided by a biological sample cartridge. Centrifugation can be in-line centrifugation where lower density material flows down the center of a revolving tube. In certain embodiments, there is one biological sample cartridge, which can contain the disposable materials used for pre-dilution sample preparation (if any), dilution buffer, a biological sample acquisition port, sample-handling components used through the dilution stage, and the like. In certain embodiments, the biological sample cartridge is simply a biological sample container, which may connect to plumbing supplied by a test cartridge.

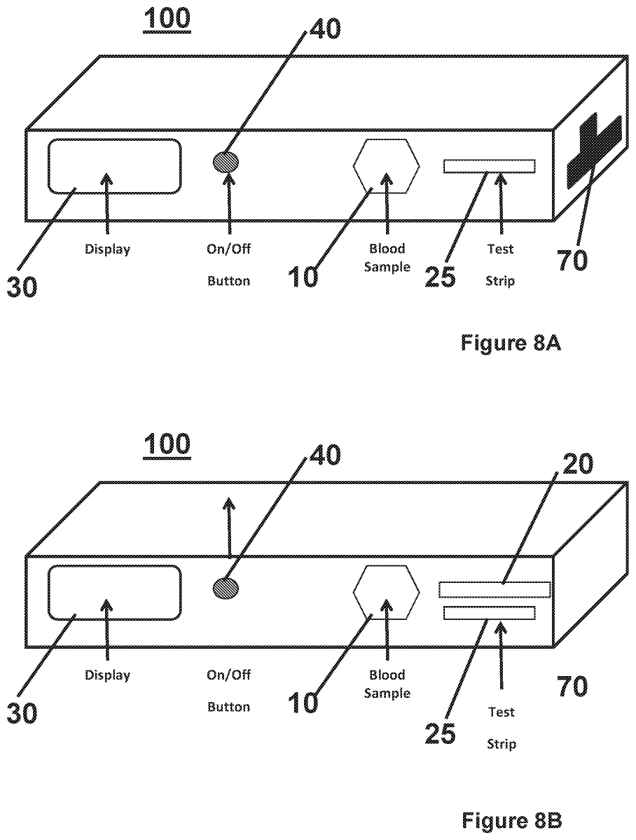

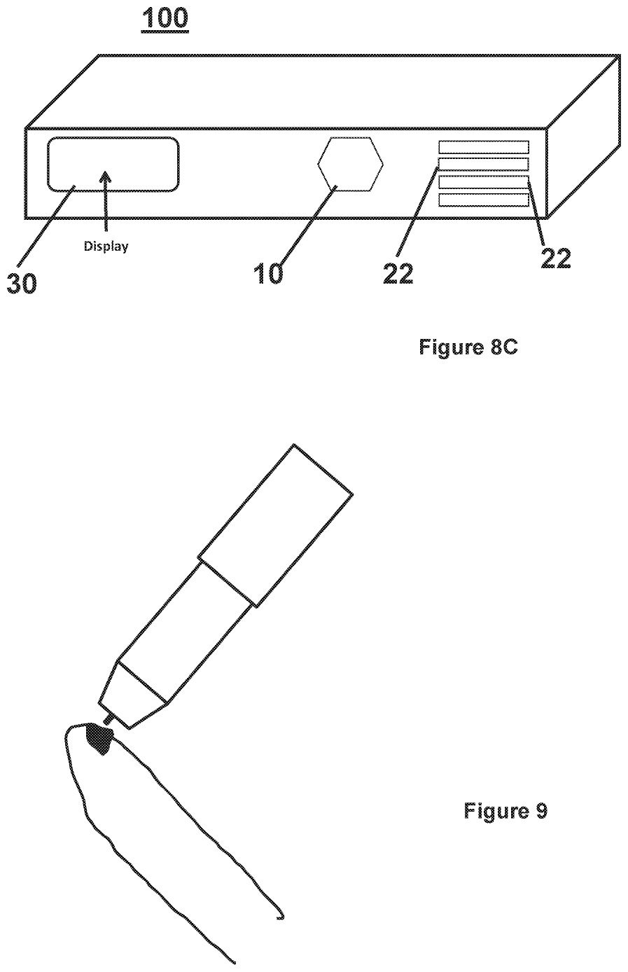

FIG. 3 shows an illustration of the point of care device. It can be a small, compact instrument comprised of for example two ports, one for the blood sample 10 (optional) and one for the targeted test cartridge 20. The shape and color of each port can be unique to prevent switching cartridges and getting an error or damaging the instrument itself. Once both cartridges are inserted properly, the instrument can initiate a pre-run to ensure all elements are present in the proper quantities for the titration assay. When the instrument is ready, a start button (e.g., 40 in FIG. 8) can illuminate indicating to an operator that the instrument is calibrated and everything is in order. The operator then simply presses the start button to commence the assay. Or, the instrument initiates the assay automatically. The results can be displayed in illustrative view screen 30, or transmitted to another device for viewing. The instrument can have connectivity ports to printers and other devices for the transfer of data by wired or wireless means. The data can be used to print results, update a patient's health record or update laboratory/hospital information systems, and the like.

In certain embodiments, the immunization test cartridge is adapted to provide a sample collection feature. For example, it may incorporate a vacutainer, or be adapted to fit a vacutainer (and for example pierce the septum of the vacutainer to draw biological sample into the test cartridge).

The input cartridges can contain circuits that save data on manufacture date or expiration date. Useful circuits include the “Touch Memory” devices from Dallas Semi-Conductors (now a subsidiary of Maxim Integrated Products), which can be adapted to connect by wire or wirelessly to the main instrument upon insertion of the test cartridges. Or, they can contain circuits for monitoring storage conditions, such as temperature. A small power source may be provided on the cartridges to power such monitoring. These circuits are adapted to convey their information to the controller. In certain embodiments, the controller can operate to re-determine an expiration date in view of monitoring data (temperature, humidity, and the like).

Assay detection can be by a single detector (e.g., light absorption detector elements, fluorescence excitation and emission monitoring, and the like) that moves relative to assay sites robotically, is directed to multiple sites with fiber optics, with small-scaled individual detectors, and the like. Or, it can be a CCD or like device that with appropriate lenses monitors all or a useful subset of assay sites simultaneously. Signal can be taken from a liquid phase, or solid phase (assay indicator adsorbed to a surface).

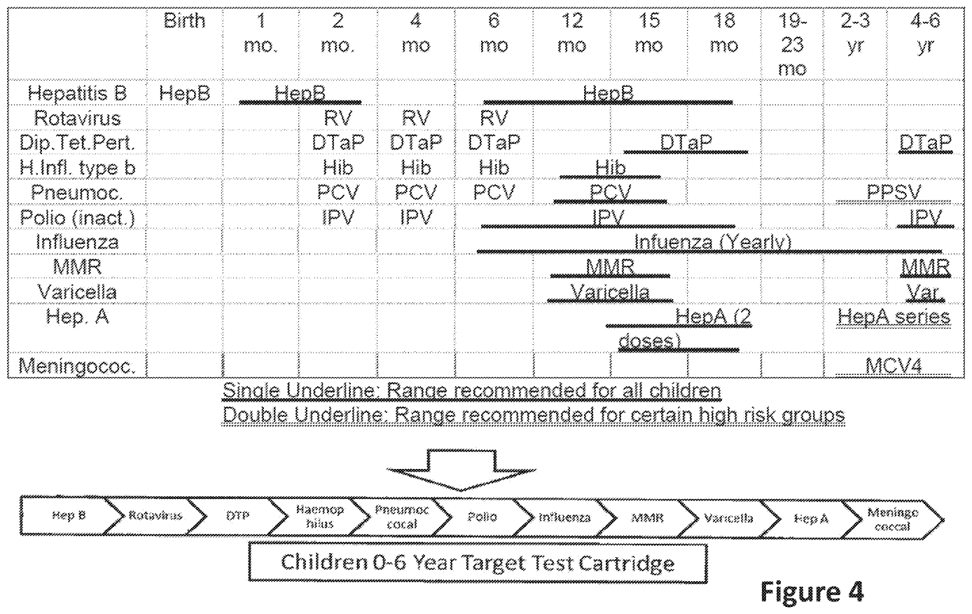

FIG. 4 shows an example of an immunization schedule, here provided by the CDC for infants. The targeted test cartridge can be the cognate equivalent of the vaccinations recommended, providing (for example) for reporting in the same order as specified in the table. In this example, eleven tests in the cartridge are housed in the test cartridge. The point of care instrument can distribute for example the specific antigen in equal parts onto assay portion per the assay protocols. The compact, targeted test cartridge can also contain a smart chip which interfaces with the point of care instrument. The smart chip can provide instrument instructions to initiate the proper test protocols, or identify the diagnostic set. The smart chip helps prevent testing errors and minimizes interactions with the operator.

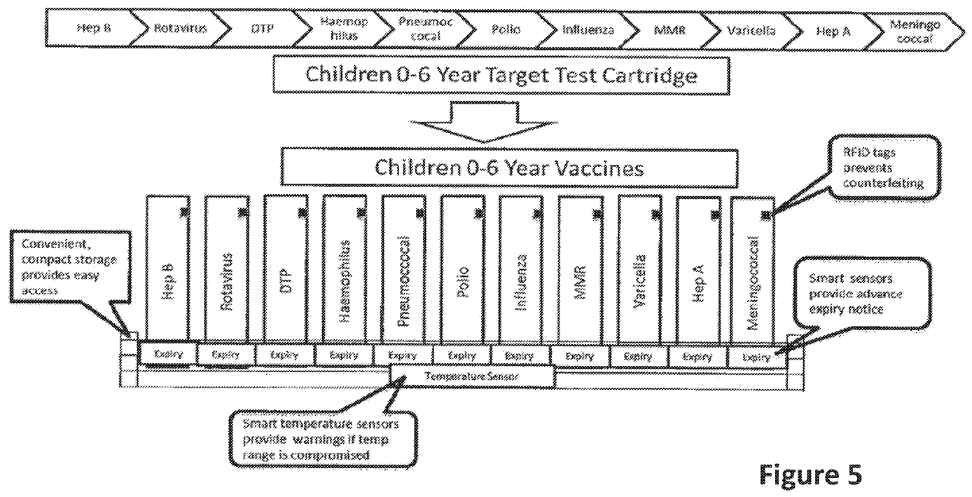

FIG. 5 shows the vaccine supply pack. The illustration is of the corresponding vaccine supply pack for infants. The vaccines (e.g., in stacks, not shown) are organized in the same sequence as reported in the output when using the infant targeted test cartridge. In the event the test results indicate a deficiency in one or more target vaccinations, the physician can simply take only those appropriate vials to administer the necessary vaccinations. The compact, organized supply pack saves space in the refrigerator and provides easy access. The vaccines can carry a simplified logo (in addition to detailed identifying information) that matches a unique logo used in the reporting of the assay data.

Smart sensors can indicate whether the vaccines have been stored outside the specified temperature range, and for how long. Smart sensors can also indicate whether a vaccine has passed its expiration date. Smart sensors can also indicate vaccine volume and provide advance warning when supply is low. The smart sensors (or connected lighting devices) can illuminate or send a signal providing the clinician advanced warning on the status and viability of the vaccine. The smart tags on each vaccine vial, whether it is a radio frequency or bar code tag, or the like, provides pedigree information to ensure the vaccine has not been compromised or chain of custody has not been broken.

In certain embodiments, the vaccine vials are fitted with IDs (which can include expiration or manufacture date data) and/or temperature monitoring sensors. The IDs can be electronic (though typically there is a printed ID), and these and any monitoring components can be communicated with by a controller (e.g., in the analytical module) wirelessly or via circuits and contacts in the vaccine supply racks. The ID's can be used to confirm that replacement vials are placed in positions that match the order provided in the analysis. In certain embodiments, the vaccine vials are shaped so that the vials can only be placed in supply pack aligned with an ID detector (e.g., bar code detector, short-range RFID reader, or the like).

In certain embodiments, vaccine supply packs are not to have replacement vaccine vials added. Thus, if a vaccine supply pack begins to run out of influenza vaccine, in certain embodiments a replacement supply pack can be ordered that contains more influenza vaccine, and relatively less of less utilized vaccines, as appropriate. Wireless communication can allow the immunization testing device, or another device, to track the inventory, utilizing sensors indicative of the presence of vaccine vials. In certain embodiments, the vaccine supply packs are adapted fit into and electronically connect to a holding rack, which can have the wireless communication hardware.

The immunization testing device or a connected input/output device can be programmed to provide safety controls. For example, a screen can announce that these are the cognate vaccines, identify an indicator of vaccination status associated with the vaccines, and seek input on which vaccines the physician or physician's assistant proposes to administer. The immunization testing device or a connected input/output device can monitor whether the correct vials are pulled (wirelessly), or insist on a validation protocol wherein IDs on the vials are confirmed to match the administration protocol. If the input administration protocol includes vaccines not strongly indicated by the test results, a warning may be outputted.

In certain embodiments, the cartridges, such as test cartridges, provide materials for negative and positive controls. Positive controls can be for example control antibody adapted to react with antigen in the positive control assay regions. Negative controls can be antibody that is not matched to a control antigen. The controller can operate to validate or reject a testing run based on the output from the controls, and can be adapted to retrieve trouble-shooting information based on the circumstances of a rejection.

Larger liquid supplies, such as dilution buffer or buffer for the immune reactions (if different) can be supplied separately from cartridges supplying antigen or the like, such as in cartridge(s) that can be utilized for several runs of the device. Alternatively, all consumables for a given run of the analytical module are provided in a test cartridge.

Illustrated in FIG. 8A is a combined immunization pack, immunization composition docking port 70. The port is adapted to fit a pack (e.g., horizontal slot) and an individual immunization composition (e.g., vertical slot) so as to align the IDs thereof for reading by the analytical module, such as by electrical contacts, Bluetooth triggered by appropriate vicinity or fit into the port, RFID triggered by appropriate vicinity, fit into the port, or alignment, bar code reader, or the like.

In certain embodiments, the analytical module weighs 20 pounds or less, or 10 pounds or less (4.536 kg or less), or 5 pounds or less, or 2 pounds or less.

In certain embodiments, the test cartridges contain one or more disease-indicative antigens. These are antigens not supplied in the corresponding vaccine, but which generate antibodies in those exposed to the native causative agent. For example, if a patient is positive for Hep B vaccine antigen and Hep B disease antigen, the physician can deduce that the patient may or may not have been vaccinated, and has probably been exposed to the virus, in which case further analysis for infection may be in order. These can be termed immune reaction source controls.

As tabulated below, the devices and methods of the disclosure can in certain embodiments:

```
TABLE
a. Provide a point of care instrument;
i. Employ microfluidics for sample and reagent handling;
ii. Employ micro-titration technology for assays;
iii. Utilize uniquely (and distinguishably) shaped and/or colored ports;
  a. Provide a biological sample cartridge
  b. Provide a targeted test cartridge
iv. Utilize a compact, small profile (for the instrument)
v. Provide rapid testing
vi. Utilize AC/DC power sources
vii. Utilize one button operation
viii. Provide low cost
ix. Provide durability
x. Provide wired connectivity
xi. Provide wireless connectivity
xii. Interface with hospital/clinical information systems. Interface with laboratory
information systems
b. Provide wired and wireless printer ports
xiii. Provide links to electronic patient records
xiv. Provide self-maintenance (e.g., via diagnostic hardware and software for the
instrument and/or the cartridges)
xvii. Provide links to smart phones, PDAs etc.
xviii. Provide biological fluid sample cartridges with
  a. Unique shape
  b. Distinguishable color
xix. Ability to supply needed sample for assay
xx. Provide targeted test cartridges with
  a. Unique shape
  b. Distinguishable color
  c. Ability to supply needed antigens for assay
  d. Chip to instruct instrument to initiate protocols
  e. Configured to meet testing requirements as set by healthcare governing
  bodies
  f. Individual chambers for each vaccine target with requisite antigen
xxi. Provide vaccine supply pack with
  a. Compact storage for vaccines
  b. Easy access
  c. Small profile saving space in refrigerator
  d. Smart sensors to indicate temperature
  e. Smart sensors to indicate expiration date
  f. Smart sensors to indicate expiration date
  g. Smart sensors to indicate vaccine supply
  h. Radio frequency tags to establish pedigree
  i. Bar code tags to establish pedigree
xxii. Provide smart app with
  a. Smart application for smart phones and computer devices
  b. Ability to track and update personal immunization records
  c. Ability to share personal immunization records
xxiii. Provide business model for selling vaccines with
    a. Companion diagnostic system for vaccines
    b. Means for identifying gaps in a person's immunization profile
    c. Administering only necessary vaccines
    d. Charging a service or handling fee for vaccine administration
    e. Charging a fee for the vaccination itself
```

In another embodiment of the disclosure, the test cartridge incorporates or operates with a test strip (which can be an array of capillaries). The test strip contains the target antigens deposited on the strip. The deposition technology can be printing, lithography, spotting or another method of deposition. The target antigens can be deposited in a series of columns representing different experimental sub-runs, such runs for one or more controls and a number of sample serial dilutions such as 1:10, 1:100, 1:100, etc. Each column has the respective antigen targets such as Hepatitis A, Rotavirus or DTP among other targets (see FIG. 7). The respective diluted sample will be deposited at the top of the column and travel down the designated channel or groove in the test strip. The deposition can contain enough fluid to move the antibodies past all the antigen depositions, or following deposition, sufficient carrier solvent is passed through the strip at the point of sample deposition to move (e.g., by capillary action) the antibodies past all the antigen depositions. The columns of the test strip are fluidically separate such that solvent flowing in one column (e.g., channel) does not carry over to another column.

At each station, the sample encounters a known antigen such as Hepatitis A. If the corresponding antibody is present in the diluted sample a binding event will occur with the target Hepatitis A antigen at the station. The binding reaction would result directly or indirectly in a color, fluorescence, optical density or like change. In certain embodiments, the detectable event is developed by passing developing agents (e.g., labeled anti-human IGG antibodies, enzyme substrates) down the columns in the same manner as used with the sample, or the like.

The remaining sample will travel to the next station, Rotavirus and if the corresponding antibody is present another binding reaction will take place designated by a direct or indirect detectable change. This will continue for all the remaining antigen stations in each column. If the corresponding antibody is not present in the diluted sample then a binding reaction will not occur and a color change (or the like) will not occur. Once all the reactions have taken place for each antigen station in every diluted sample column a number of stations will have changed color. One then would read the results across each target antigen row such as Hepatitis A and plot the results on a dose response curve. If the dose response curve matches the target established by the governing healthcare agency such as the CDC (Centers for Disease Control) in the US then the patient does not need additional vaccinations. If the dose response curves do not match then the patient would be advised by their physician to get a vaccination only for the vaccines that are in question. The establishment of the patient's immunity status is thereby called the immunoprofile. Once the necessary vaccines have been updated the patient's immunoprofile can also be updated and disseminated to medical records, patient records and insurance records among others.

The test strip is configured with the necessary immunization tests per the healthcare guidelines of the specific country for the appropriate group by age, gender and life changing event among others. The test strip 25 can be inserted into the point of care diagnostic instrument (FIGS. 8A and 8B) in the same manner as the aforementioned targeted test cartridge. The blood sample cartridge will remain the same and serve the same function as prior which is to supply the instrument with the patient's blood sample. The blood sample cartridge can have a unique shape from the test strip so as to be differentiated and cannot be mistaken for each other.

As illustrated in FIG. 8C, there can be multiple items 22, which can be immunization testing cartridges or testing strips or combinations. Similarly, there can be multiple blood sample ports 10—or there can be one or more blood sample ports 10 that shuttle blood sample to multiple items 22. In this way, multiple immunization tests can be conducted from different patients simultaneously, or separate immunization tests for the same patient can be conducted simultaneously.

In another embodiment of the disclosure, the point of care diagnostic instrument is a handheld sampling device which can be a finger prick device (FIG. 9). In this design the sample cartridge is eliminated as the sample is drawn directly from the skin into the device (test cartridge) similar to current blood glucose meters. The sample is prepped as needed using an anti-coagulant to facilitate the serial dilution process described earlier. Once the serial dilution has been complete per the guidelines of the prescribed test, the appropriate dilutions are then assayed. The assay can be via a test strip such as described earlier. For example, the sampling device can incorporate micro-capillaries with the deposited antigens. The hand held version of the point of care instrument would then read the results in the same manner as described above and determine based upon the patient's immunoprofile if any vaccinations are needed to address any identified gaps. Once the necessary vaccines have been updated the patient's immunoprofile can also be updated and disseminated to medical records, patient records and insurance records among others.

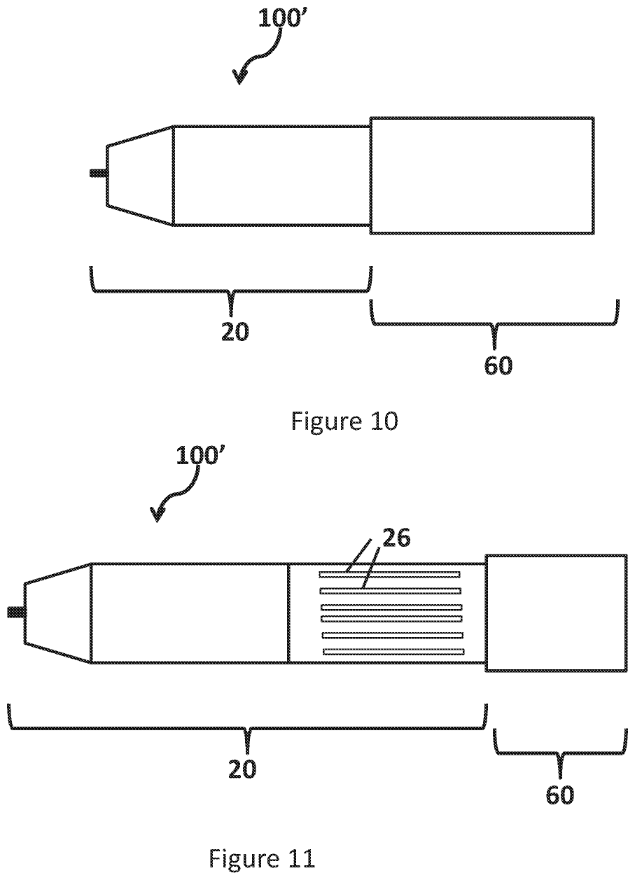

The handheld sampling device can be reusable with disposable test strips similar to the point of care instrument, or disposable. In one embodiment, a portion comprises the test cartridge 20, and is made up of elements for which it is more economically feasible to dispose on each use, and which includes the fluid-handling components. Electrical, mechanical, fiber optic and the like systems can be comprised within this test cartridge, but more expensive components can be in a separate section, such as controller 50 in the illustration of FIG. 10, which can be reversibly connected to the test cartridge. In certain embodiments, the controller 50 is separate from the test cartridge 20 and connects wirelessly or by wire (e.g., USB, or mini USB).

In certain embodiments, the handheld sampling device incorporates viewing windows 26 or the like (FIG. 11) so that the columns of the test strip can be viewed by the user. The windows can have markings to indicate the vaccine antigen alignment, and the various columns (1:1,000; positive control, etc.).

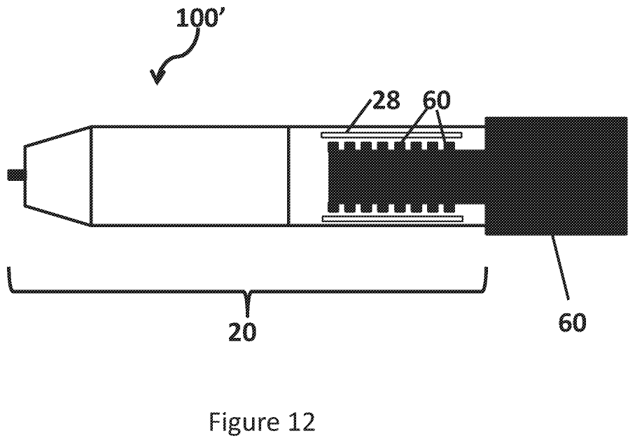

In certain embodiments, such as illustrated in the cross-section of FIG. 12, the controller 50 cameras or fiber optic outputs (optical elements) that insert (with fluid segregation) into the test cartridge so that detector elements are not disposable. The optical elements 60 are aligned with the antigen-bound portions of columns 28 of a test strip.

The handheld device is all inclusive able to perform all functions of the point of care instrument. The point of care instrument can also be connected to a computer or the like using for example an USB type connector to transfer and display results. The handheld will also have the same functions as the point of care instrument described earlier but in a portable form to offer convenience and greater value.

It should be noted that the test cartridge illustrated for the handheld fluid sampling device can be the test cartridge utilized in the device of FIG. 3, or of FIG. 8B.

The disclosure includes methods comprising the functionality described for any embodiment of the testing device or of the periphery devices operative with the testing device.

The immunization testing device can be portable (such as hand-held), or non-portable.

The disclosure includes at least the following numbered embodiments:
A. An point-of-care testing device for testing a biological sample from a subject for immunization/sensitization status comprising: ▪an analytical module adapted to make one or two or more dilutions of a fluid that is or is derived from the biological sample, contact said dilutions with separate replicates of vaccine- or sensitization-indicative antigens so as to generate a signals indicative of the amount of antigen-reactive immune molecules in the biological sample dilutions.
B. The immunization testing device of embodiment A for testing a biological sample from a subject comprising, the analytical module comprising ▪a controller, ▪an data output device, and ▪one or more input ports having a conjugate input comprising an immunization test cartridge, wherein the analytical module is adapted to receive an ID from an immunization test cartridge, wherein the controller is adapted to operate the analytical module to make the two or more dilutions of a fluid that is or is derived from the biological sample, contact said dilutions with the separate replicates of vaccine- or sensitization-indicative antigens so as to generate a signals indicative of the amount of antigen-reactive immune molecules in the fluid dilutions, interpret the received ID to identify one of a pre-set plurality of available immunization test cartridges, and to utilize the generated signals and the immunization test cartridge to output a report on the immune status of the subject with respect to an array of separate vaccine- or sensitization-indicative antigens.
C. The immunization testing device of embodiment A or B, wherein any ports present are shaped to accept their conjugate input and not accept the conjugate inputs of other ports present.
D. The immunization testing device of embodiment A, B or C, wherein a controller is adapted to operate with ▪a collection of immunization test cartridges comprising reagents for testing immune status against an array of vaccine- or sensitization-indicative antigens, the collection including two or more cartridges for testing separate arrays of vaccine- or sensitization-indicative antigens, the separate arrays adapted for use with separate patient populations, the cartridges having IDs that are distinctive of the separate arrays, wherein the testing device reads the ID of a given utilized test cartridge and presents an output report correlating the vaccination status results with the respective vaccine- or sensitization-indicative antigens based on the read ID.
E. The immunization testing device of one of the foregoing embodiments, wherein the controller is adapted to operate with ▪packs of immunization compositions, separate packs matching the separate arrays of the immunization test cartridges, the distinct immunization compositions identifiably spatially segregated on the packs, the packs having IDs that are distinctive of the separate arrays of immunization compositions, wherein the immunization testing device comprises an immunization pack ID reader, and wherein after presenting an output report the controller compares the ID of a presented pack with the ID of the utilized test cartridge to confirm that the cognate pack has been presented.
F. The immunization testing device of embodiment one of the foregoing embodiments, wherein the compositions have distinctive IDs, and wherein the immunization testing device comprises an immunization composition ID reader, and wherein after presenting an output report the controller compares an ID of a presented immunization composition with the output report data to confirm that the immunization composition matches the immunization composition called for by the immune status report.
G. The immunization testing device of embodiment one of the foregoing embodiments, wherein the controller is adapted to operate with ▪packs of immunization compositions, separate packs matching the separate arrays of the immunization test cartridges, wherein the immunization testing device comprises an immunization composition ID reader, and wherein after presenting an output report the controller compares an ID of a presented immunization composition with the output report data to confirm that the immunization composition matches the tested vaccine-indicative antigens.
H. The immunization testing device of embodiment F or G, wherein the controller further confirms that the presented immunization composition matches a vaccine-indicative antigen found to have a deficient immune response.
I. The immunization testing device of one of the foregoing embodiments, in the form of a handheld sampling device.
J. The immunization testing device of one of the foregoing embodiments, wherein the vaccine indicative antigens are arrayed on a test strip adapted to contact the dilutions by flow of the dilution material through columns of the test strip so as to serially contact the antigens.
K. The immunization testing device of embodiment J, wherein the test strips are incorporated into the immunization test cartridges.
L. A collection of immunization test cartridges comprising reagents for testing immune status against an array of vaccine- or sensitization-indicative antigens, the collection including two or more cartridges for testing separate arrays of vaccine- or sensitization-indicative antigens, the separate arrays adapted for use with separate patient populations, the cartridges having IDs that are distinctive of the separate arrays.
M. A kit comprising ▪a collection of immunization test cartridges of embodiment L, ▪packs of immunization compositions, separate packs matching the separate arrays of the immunization test cartridges, the distinct immunization compositions identifiably spatially segregated on the packs, the packs having IDs that are distinctive of the separate arrays of immunization compositions, the collections and packs adapted to be operative with an analytical module that utilizes the immunization test cartridges to provide subject immune statuses for the antigens of given immunization test cartridges, and which compares the ID of a utilized immunization test cartridge and that of a presented pack to confirm that the cognate pack has been presented.
N. The kit of embodiment M, wherein the immunization composition packs comprise a temperature sensor, electronic memory for tracking temperature from the sensor over time, and wherein the packs are adapted to communicate the temperature tracking to the analytical module.
O. The kit of embodiment M or N, wherein the immunization test cartridges are in the form of handheld sampling devices.
P. A method of operating the testing device of an embodiment A-K comprising operating the testing device with a biological sample from a patient utilizing an immunization test cartridge, and thereby outputting a report on the immune status of the subject with respect to an array of separate vaccine- or sensitization-indicative antigens.
Q. A method of operating the testing device of an embodiment A-K, comprising ▪operating the testing device with a biological sample from a patient utilizing a said immunization test cartridge, and thereby outputting a report on the immune status of the subject with respect to an array of separate vaccine-indicative antigens; ▪presenting a said immunization pack to the immunization pack ID reader to generate output from the testing device confirming or negating that the immunization pack is the cognate of the test cartridge.
R. The method of embodiment Q, wherein the controller obtains from the immunization pack information on immunization stock, calculates the further utilization implied by the immune status report, and, if needed based on this data, generates (i) a report identifying vaccine restocking needs or (ii) a purchase order to a vaccine supplier.
S. A method of operating the testing device of an embodiment A-K, comprising ▪operating the testing device with a biological sample from a patient utilizing a said immunization test cartridge, and thereby outputting a report on the immune status of the subject with respect to an array of separate vaccine-indicative antigens; and ▪presenting a said immunization composition to the immunization composition ID reader to generate output from the testing device confirming or negating that the immunization composition is the immunization composition called for by the immune status report.
T. A method of operating the testing device of an embodiment A-K, comprising ▪operating the testing device with a biological sample from a patient utilizing a said immunization test cartridge, and thereby outputting a report on the immune status of the subject with respect to an array of separate vaccine-indicative antigens; and ▪presenting a said immunization composition to the immunization composition ID reader to generate output from the testing device confirming or negating that the immunization composition is the immunization composition called for by the immune status report.
U. The method of an embodiment P-T, wherein the analytical module is comprised in a handheld sampling device.
V. The method of an embodiment P-T, where the method is conducted at the point-of-care.

According to one aspect of the present disclosure, a method for testing a biological sample from a subject for immunization or sensitization status is provided. The method comprises: in a label-based assay contacting an undiluted biological sample obtained from the subject with a plurality of vaccination-indicative antigens or sensitization-indicative antigens disposed on a test strip or cartridge; detecting the presence or absence of a threshold amount of a plurality of immune mediators reactive to the antigens present in the biological sample based on the contacting, thereby determining immunization or sensitization status with respect to the antigens; and generating a report on which of the one or more types of vaccination- or sensitization-indicative antigens correspond with a threshold amount of the plurality of antigen-reactive immune mediators present in the biological sample.

In some embodiments, the method may be performed at a point-of-care.

In certain embodiments, the method may be performed at a point-of-assessment.

In some embodiments, the method may be performed at a point-of-diagnosis.

In certain embodiments, the contacting may be conducted in one or more immune-responsive sensors, and the detecting may comprise signals from the sensors, wherein the signal size for a given vaccination- or sensitization-indicative antigen is indicative of whether the threshold amount is present.

In some embodiments, the detecting may be performed in panel with multiple tests simultaneously or in sequence.

In certain embodiments, the detecting may comprise one or more assays selected from the group consisting of.
(a) an enzyme assay
(b) an ELISA assay
(c) a fluorogenic reporter assay;
(d) an electrochemiluminescent assay;
(e) a sensor-based assay;
(f) a competitive assay; and
(g) a noncompetitive assay.

In certain embodiments, the label-based assay may utilize one or more labels selected from the group consisting of:
(a) a fluorescence label;
(b) a fluorogenic label;
(c) an electrochemical label;
(d) an electrochemical-luminescent label;
(e) a microparticle label; and
(f) a nanoparticle label.

In some embodiments, the sensor-based assay may utilize one or more sensors selected from the group consisting of:
(a) a chemical sensor;
(b) an electrical sensor;
(c) an electrochemical sensor;
(d) a paramagnetic sensor; and
(e) an optical sensor.

In certain embodiments, the contacting and detecting may be performed on a microfluidics lab-on-a-chip.

In some embodiments, the contacting and detecting may be performed using the test strip or cartridge and a detector or analyzer.

In certain embodiments, the detector or analyzer may be a point-of-care detector or point-of-care analyzer.

In some embodiments, the assay may be performed using the test strip or cartridge and a handheld device.

In certain embodiments, the handheld device may be a point-of-care handheld device.

In certain embodiments, the biological sample comprises blood, sera, lymph fluid, urine, tears, or saliva.

In certain embodiments, the plurality of antigen-reactive immune mediators includes antibodies.

In some embodiments, the test strip or cartridge contains a vaccination-indicative antigen or sensitization-indicative antigen which may be selected for a specific patient population.

In certain embodiments, the patient population may be classified based on age, gender, location, or any combination thereof.

In certain embodiments, the plurality of antigen-reactive immune mediators may include antibodies.

According to yet another aspect of the present disclosure, a method for testing a biological sample from a subject for immunization or sensitization status is provided. The method comprises: in a label-based assay contacting a biological sample obtained from the subject with a plurality of vaccination-indicative antigens or sensitization-indicative antigens disposed on a test strip or cartridge; and with a detector or analyzer, detecting the presence or absence of a threshold amount of a plurality of immune mediators reactive to said antigens present in the biological sample based on the contacting, thereby determining immunization or sensitization status with respect to the antigens.

In certain aspects, the method may further include generating a report on which of the one or more types of vaccination- or sensitization-indicative antigens correspond with a threshold amount of the plurality of antigen-reactive immune mediators present in the biological sample.

In some aspects, the biological sample may be an undiluted biological sample.

In certain aspects, detecting may comprise one or more assays selected from the group consisting of:
(a) an enzyme assay;
(b) an ELISA assay;
(c) a fluorogenic reporter assay;
(d) an electrochemiluminescent assay;
(e) a sensor based assay;
(f) a competitive assay; and
(g) a noncompetitive assay.

In certain aspects, the biological sample may comprise blood, sera, lymph fluid, urine, tears, or saliva.

According to yet another aspect of the present disclosure, a method for testing a biological sample from a subject for immunization or sensitization status is provided. The method comprises: in a label-based assay contacting a biological sample obtained from the subject with a plurality of vaccination-indicative antigens or sensitization-indicative antigens disposed on a test strip or cartridge that contains a vaccination-indicative antigen or sensitization-indicative antigen selected for a specific patient population classified based on age, gender, location, or any combination thereof, and detecting the presence or absence of a threshold amount of a plurality of immune mediators reactive to the antigens present in the biological sample based on the contacting, thereby determining immunization or sensitization status with respect to the antigens.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

1. An immunization method comprising administration of one or more vaccines to a subject identified as having a deficiency in immunization or sensitization status to one or more pathogens wherein the status is determined using a diagnostic method comprising:
(i) contacting a diluted or undiluted biological sample obtained from the subject with a device comprising a diagnostic set of reagents for simultaneously conducting label-based immunoassays wherein the label-based assay utilizes one or more labels selected from the group consisting of: a fluorescence label; a fluorogenic label; an electrochemical-luminescent label; a microparticle label; and a nanoparticle label and wherein said immunoassays include a plurality of vaccination-indicative antigens or sensitization-indicative antigens derived from multiple pathogens disposed on a test strip or cartridge;
(ii) detecting the presence or absence of a threshold amount of a plurality of antigen reactive antibodies present in the biological sample based on the contacting, wherein the contacting of step (i) is performed on a microfluidics lab-on-a-chip, and wherein the detecting comprises use of one or more assays selected from the group consisting of an enzyme assay; an ELISA assay; a fluorogenic reporter assay; an electro-chemiluminescent assay; a competitive assay; a noncompetitive assay; and a sensor-based assay wherein said sensor-based assay utilizes one or more sensors selected from the group consisting of a chemical sensor; a paramagnetic sensor; and an optical sensor;
(iii) comparing established standard levels of antibodies indicative of protective immunization to the threshold amount of a plurality of antigen reactive antibodies present in the biological sample;
wherein detection of lower levels of antigen reactive antibodies as compared to the established standard levels of antibodies indicative of protective immunization indicates a deficiency in immunization or sensitization status; and
(iv) where a deficiency in immunization or sensitization status is indicated, one or more vaccines are administered to the subject.

2. The method of claim 1, wherein the diagnostic method is performed at a point-of-care.

3. The method of claim 1, wherein the diagnostic method is performed at a point-of-assessment.

4. The method of claim 1, wherein the diagnostic method is performed at a point-of-diagnosis.

5. The method of claim 1, wherein the contacting is conducted in one or more immune-responsive sensors, and the detecting comprises signals from the sensors, wherein the signal size for a given vaccination- or sensitization-indicative antigen is indicative of whether the threshold amount is present.

6. The method of claim 1, wherein the detecting is performed in panel with multiple tests simultaneously or in sequence.

7. The method of claim 1, wherein the contacting and detecting is performed using the test strip or cartridge and a detector or analyzer.

8. The method of claim 7, wherein the detector or analyzer is a point-of-care detector or point-of-care analyzer.

9. The method of claim 1, wherein the diagnostic assay is performed using a test strip or cartridge and a handheld device.

10. The method of claim 9, wherein the handheld device is a point-of-care handheld device.

11. The method of claim 1, wherein the biological sample comprises blood, sera, lymph fluid, urine, tears, or saliva.

12. The method of claim 1, wherein the test strip or cartridge contains one or more vaccination-indicative antigens or sensitization-indicative antigens which are selected for a specific patient population.

13. The method of claim 12, wherein the patient population is classified based on age, gender, location, or any combination thereof.

14. The method of claim 1, wherein a dose response curve is deduced based on the amount of antigen-reactive antibodies detected in the diluted sample.

15. The method of claim 1, wherein the plurality of vaccination-indicative antigens or sensitization-indicative antigens are derived from the group of pathogens consisting of measles, mumps, rubella, diphtheria, tetanus, pertussis, polio, hepatitis A, hepatitis B, hepatitis C, rotovirus, haemophilus, pneumococcal, influenza, varicella and meningococcal.

16. A method for testing a biological sample from a subject for immunization or sensitization status comprising:
in a label-based assay contacting an undiluted biological sample obtained from the subject with a plurality of vaccination-indicative antigens or sensitization-indicative antigens disposed on a test strip or cartridge;
detecting the presence or absence of a threshold amount of a plurality of immune mediators reactive to the antigens present in the biological sample based on the contacting, thereby determining immunization or sensitization status with respect to the antigens; and
generating a report on which of the one or more types of vaccination- or sensitization-indicative antigens correspond with a threshold amount of the plurality of antigen-reactive immune mediators present in the biological sample.

17. A method for testing a biological sample from a subject for immunization or sensitization status comprising:
in a label-based assay contacting a biological sample obtained from the subject with a plurality of vaccination-indicative antigens or sensitization-indicative antigens disposed on a test strip or cartridge; and
with a detector or analyzer, detecting the presence or absence of a threshold amount of a plurality of immune mediators reactive to said antigens present in the biological sample based on the contacting, thereby determining immunization or sensitization status with respect to the antigens.
